(19) United States
(12) Patent Application Publication
(10) Pub. No.: US 2026/0325130 A1
(43) Pub. Date: Oct. 22, 2026

(54) SUTURE APPARATUS AND AUTOMATED MANUFACTURING PROCESS

(71) Applicant: Arthrex, Inc., Naples, FL (US)

(72) Inventors: Thomas Dooney, Naples, FL (US); Nirav Patel, Naples, FL (US); Nathanael Gamso, Naples, FL (US); John Seyler, Naples, FL (US); Derek C. Sullivan, Naples, FL (US); Colin Baker, Naples, FL (US); Ben Gibson, Naples, FL (US); John Sodeika, Naples, FL (US)

(73) Assignee: Arthrex, Inc., Naples, FL (US)

(21) Appl. No.: 19/399,811

(22) Filed: Nov. 25, 2025

Related U.S. Application Data
(60) Provisional application No. 63/725,177, filed on Nov. 26, 2024.
(60) Provisional application No. 63/736,702, filed on Dec. 20, 2024.

(51) Int. Cl. A61B 17/06; A61B 17/00

(52) U.S. Cl. CPC A61B 17/06166; A61B 2017/00526; A61B 2017/06171

(57) ABSTRACT

A suture is formed from a plurality of continuous strands and includes a continuous length of a carrier braid extending from a first end portion to a second end portion. At least one window includes a plurality of legs bifurcated from the continuous strands defining a window length.

### CROSS-REFERENCE TO RELATED APPLICATIONS

This application claims priority under 35 U.S.C. § 119(e) and the benefit of U.S. Provisional Application No. 63/725,177 entitled SUTURE APPARATUS AND AUTOMATED MANUFACTURING PROCESS, filed on Nov. 26, 2024, by Dooney, et al., and 63/736,702 entitled SUTURE APPARATUS AND AUTOMATED MANUFACTURING PROCESS, filed on Dec. 20, 2024, by Dooney, et al., the entire disclosures of which are incorporated herein by reference.

### BACKGROUND

The present disclosure generally relates to a suture manufactured with an automated or automation-assisted manufacturing process and, more particularly, to a suture formed from continuous yarns or strands, including a plurality of in-line features integrated through the manufacturing process. In general, advancements in suture technology continue to improve patient outcomes by facilitating various improved or advanced techniques for surgeons. However, in various cases, integrating features into sutures may require significant manual labor and skill to limit variation and ensure the resulting sutures and assemblies are consistently delivered with a high level of quality. The disclosure provides for a variety of features that may be implemented into a suture through one or more automated or automation-assisted processes that may limit manual labor to improve quality and limit variations in manufacturing.

### SUMMARY

The disclosure generally provides for a suture formed from a plurality of continuous strands or yarns of material that are interlaced to incorporate a plurality of features along a continuous length. The features may be formed with automated equipment that may direct each of the yarns in various interlaced patterns with a plurality of carriers. In operation, the path of the carriers may be controlled along programmable tracks directing horn gears to adjust the interlaced configuration. Based on variations in the tracks and the positions of the carriers in slots on each of the horn gears, the resulting features may include one or more transition sections that may vary the cross-section of the suture from a single uniform shape having a first cross-section to different shapes or bifurcated segments having different cross-sections. By implementing features in coordinated segments along the continuous length of the suture, the disclosure may provide for a suture construct with various features incorporated along the length produced with minimal manual assembly.

In various implementations, the disclosure provides for a suture (e.g., a tissue suture or connecting suture) braided or otherwise formed from a plurality of continuous strands that form a continuous length of a carrier braid extending from a first end portion to a second end portion. Along the continuous length, the continuous strands may be transitioned in cross-section from, for example, a round or rectangular cross-section to a flat or bifurcated separate cross-sections. Each of these variations in cross-section may be referred to as transitions or transition sections of the suture formed by the continuous strands. By arranging these transitions and the corresponding features in a defined order and/or spacing along the continuous length, the disclosure may provide for the tissue suture to be readily implemented in a surgical assembly or construct. Specific examples of such features, tissue sutures, and resulting constructs are discussed in the following detailed description along with corresponding methods of manufacturing.

These and other features, objects and advantages of the present disclosure will become apparent upon reading the following description thereof together with reference to the accompanying drawings.

### BRIEF DESCRIPTION OF THE DRAWINGS

FIG. 1 is a schematic assembly view of an anchor construct including a suture having a plurality of features incorporated in a continuous structure;

FIG. 2 is a detailed pictorial view of a suture demonstrating a plurality of features incorporated along a continuous length;

FIG. 3A is a schematic view of a suture including a plurality of features formed by transitions along a continuous length;

FIG. 3B is a schematic assembly view of the suture of FIG. 3A in an unmodified form;

FIG. 3C is a detailed assembly view of the anchor construct introduced in FIG. 1 demonstrating the assembly or threading of a shuttle suture;

FIG. 4A is a detailed pictorial view of a suture demonstrating a plurality of windows;

FIG. 4B is a cross-sectional view of the suture demonstrated in FIG. 4A, sectioned along line 1-1;

FIG. 4C is a detailed schematic view of a window of the suture demonstrated in FIG. 4A;

FIG. 5A is a schematic diagram of a suture incorporated along a continuous length in the form of a transition section;

FIG. 5B is a cross-sectional view of the transition section demonstrated in FIG. 5A, along section line II-II;

FIG. 5C is a schematic diagram of a suture demonstrating a feature incorporated along a continuous length demonstrating a single transition in cross-section;

FIG. 6A is an assembly diagram demonstrating a step for implementing a tissue suture with a knotless anchor construct;

FIG. 6B is an assembly diagram demonstrating a step for implementing a tissue suture with a knotless anchor construct;

FIG. 6C is an assembly diagram demonstrating a step for implementing a tissue suture with a knotless anchor construct;

FIG. 6D is an assembly diagram demonstrating a step for implementing a tissue suture with a knotless anchor construct;

FIG. 6E is an assembly diagram demonstrating a step for implementing a tissue suture with a knotless anchor construct in accordance with the disclosure;

FIG. 7 is a schematic view of a suture including a plurality of features formed by transitions along a length;

FIG. 8A demonstrates a picture of a suture loop formed by a suture including a plurality of transition sections;

FIG. 8B demonstrates a schematic diagram of a suture loop formed from a spliced suture construct;

FIG. 8C demonstrates a schematic diagram a suture loop formed from a spliced suture construct;

FIG. 9A demonstrates a process diagram demonstrating an assembly method for a suture loop formed from a spliced suture construct;

FIG. 9B demonstrates a process diagram demonstrating an assembly method for a suture loop formed from a spliced suture construct;

FIG. 9C demonstrates a process diagram demonstrating an assembly method for a suture loop formed from a spliced suture construct;

FIG. 9D demonstrates a process diagram demonstrating an assembly method for a suture loop formed from a spliced suture construct;

FIG. 9E demonstrates a process diagram demonstrating an assembly method for a suture loop formed from a spliced suture construct;

FIG. 10 is a schematic diagram of a suture comprising a plurality of integrated features positioned at predetermined positions along a length; and

FIG. 11 is a flowchart demonstrating a manufacturing or fabrication process for a suture construct described in reference to the suture of FIG. 10.

### DETAILED DESCRIPTION

In the following description, reference is made to the accompanying drawings, which show specific implementations that may be practiced. Wherever possible, the same reference numbers will be used throughout the drawings to refer to the same or like parts. It is to be understood that other implementations may be utilized and structural and functional changes may be made without departing from the scope of this disclosure.

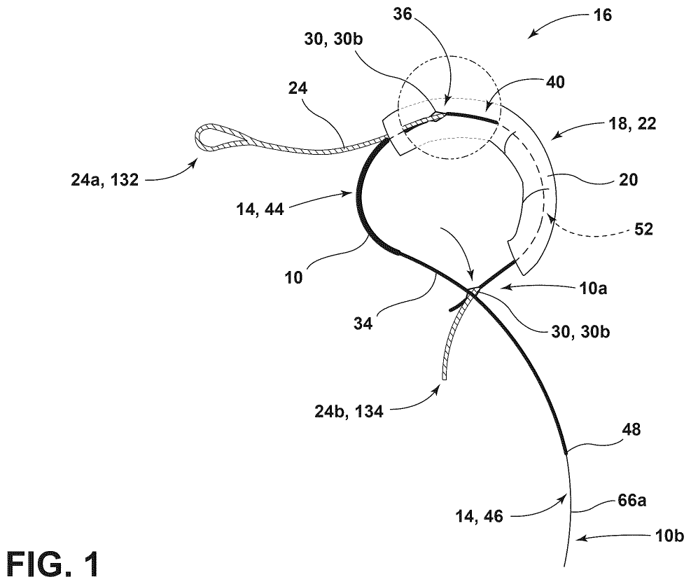

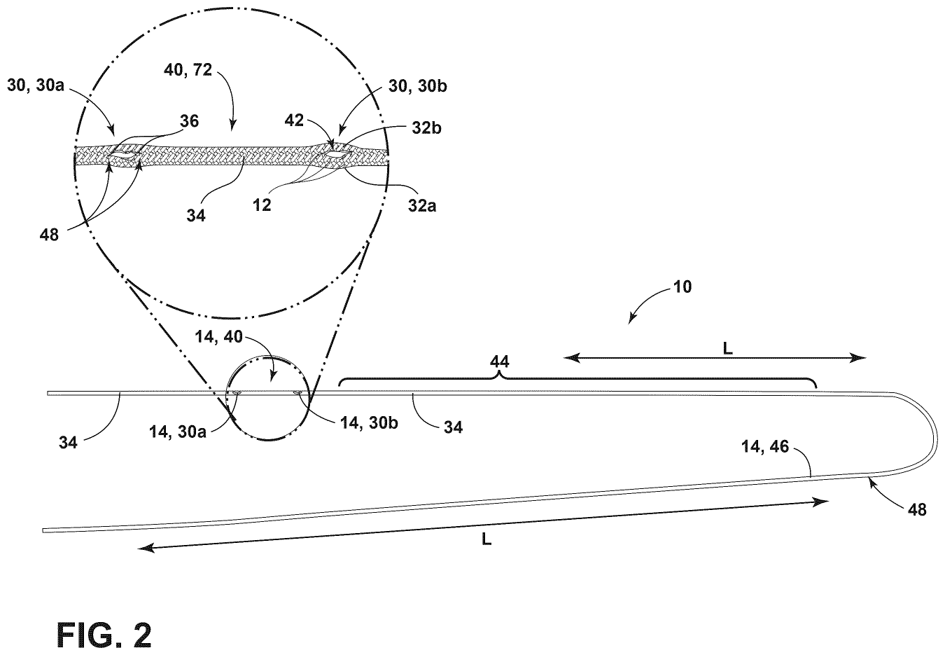

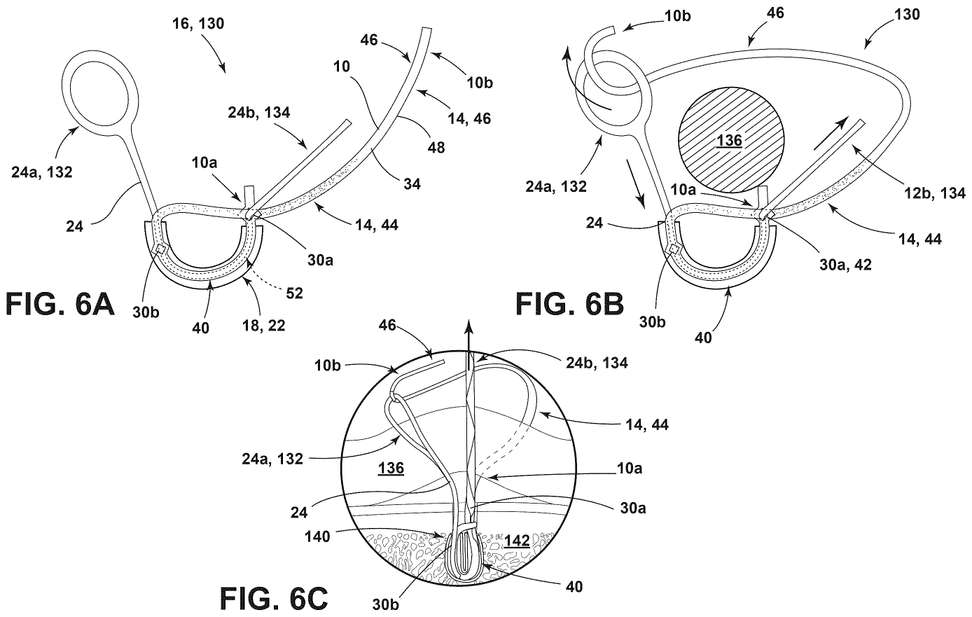

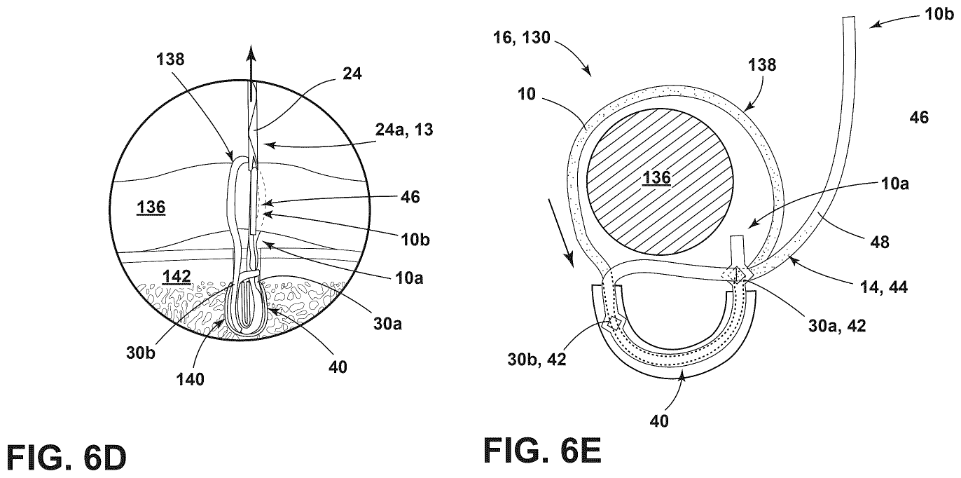

As demonstrated in FIGS. 1 and 2, the disclosure generally provides for a suture 10 that may be implemented in a variety of surgical constructs and/or assemblies. In general, the suture 10 may be formed form a plurality of continuous yarns or strands 12 and form a plurality of integrated features 14 produced by controlling the intertwined paths of the strands 12 in various configurations. In the example shown in FIG. 1, the suture 10 is demonstrated in a surgical assembly 16 comprising an anchor construct 18. As shown, the anchor construct 18 may include an anchor body 20, which may be in the form of a soft anchor 22 (as shown) or various other anchors, including rigid or threaded anchors. The example shown in FIG. 1 further includes a passing or shuttle suture 24 that may allow the tissue suture 10 to be employed in a knotless configuration. Further details of the operation of the tissue suture 10 in the anchor construct 18 are described in reference to FIGS. 6A-6E of the following description.

In various implementations, the features 14 incorporated along the continuous length L of the suture 10 may be implemented by automated processes resulting from transitioning the intertwined paths of the strands 12 in various configurations. By adjusting the intertwined paths of the strands 12, the disclosure may provide for improved manufacture and assembly of the anchor construct 18 as well as various surgical assemblies 16. For example, the integrated features 14 formed by the intertwined strands 12 may improve or replace various post-processing steps including manually connected features with automated or machine-assisted operations. In this way, the disclosure may provide for a variety of implementations of sutures and corresponding surgical assemblies that may improve patient outcomes while limiting manufacturing time and expense.

Still referring to FIGS. 1 and 2, in the example shown, the integrated features 14 formed by the continuous strands 12 along the length L of the suture 10 include a plurality of windows 30 braided or formed from the continuous strands 12. As shown, the windows 30 may be formed by a plurality of legs 32, including a first leg 32a and a second leg 32b, which may be bifurcated from a common carrier braid 34 and formed from the continuous strands 12 along the length L. In various implementations, the windows 30 may correspond to integrated features 14 that may provide for the attachment of the suture 10 to various additional sutures or constructs. In some implementations, the windows 30 may additionally or alternatively provide for an opening or access region 36 centrally within a cross-section of the suture 10 formed by the intertwined continuous strands 12. In this way, one or more sutures or materials may be threaded or fed into the suture 10 via a transverse opening extending through the access region. For clarity, the term strand may be used in this disclosure to describe various thread-like structures that may correspond to various fibers comprising one or more multifilament or monofilament yarns. Further, the carrier 34 or carrier braid may correspond to a combination of the strands 12 interlinked along the length L of the suture 10, particularly between features introduced by transitions or transition sections 48.

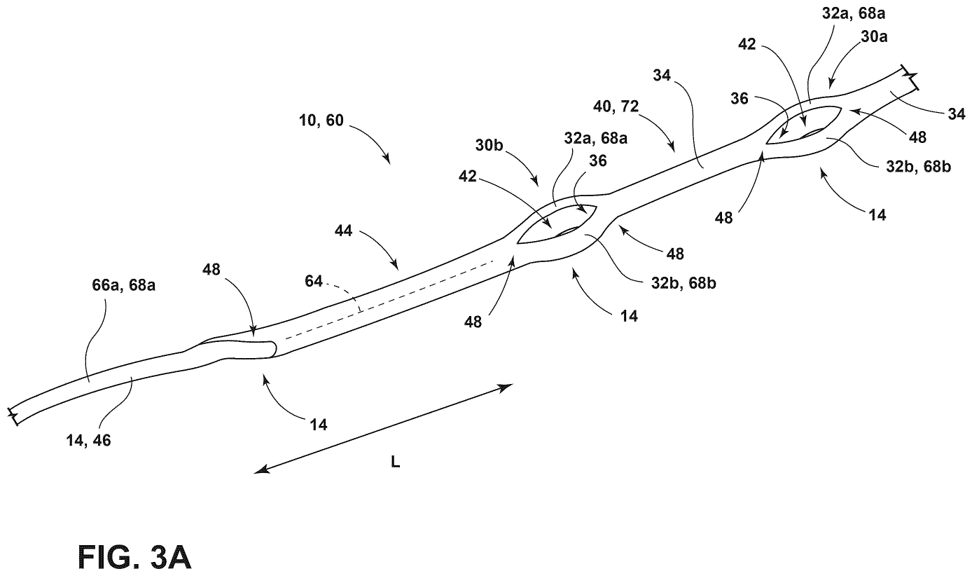

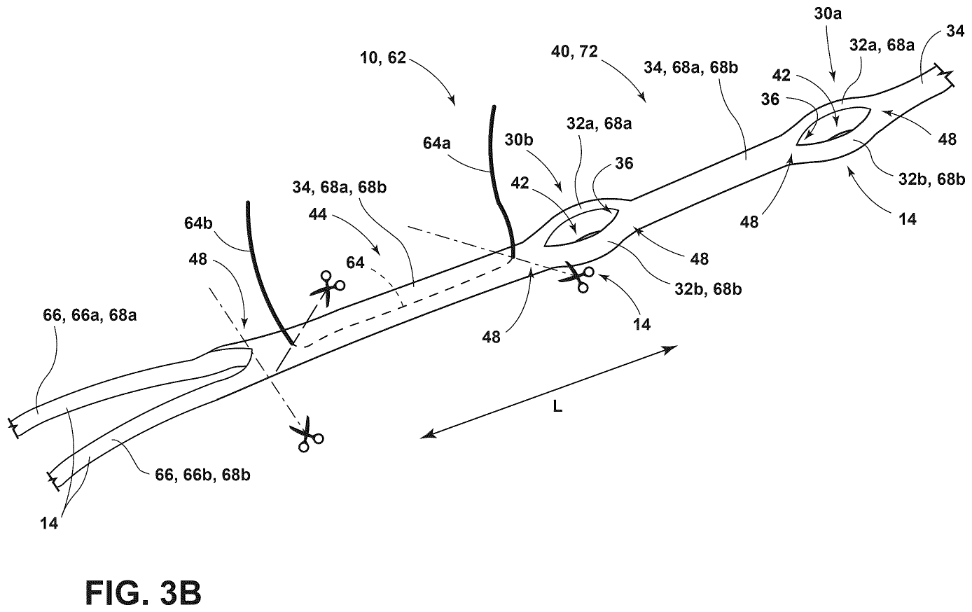

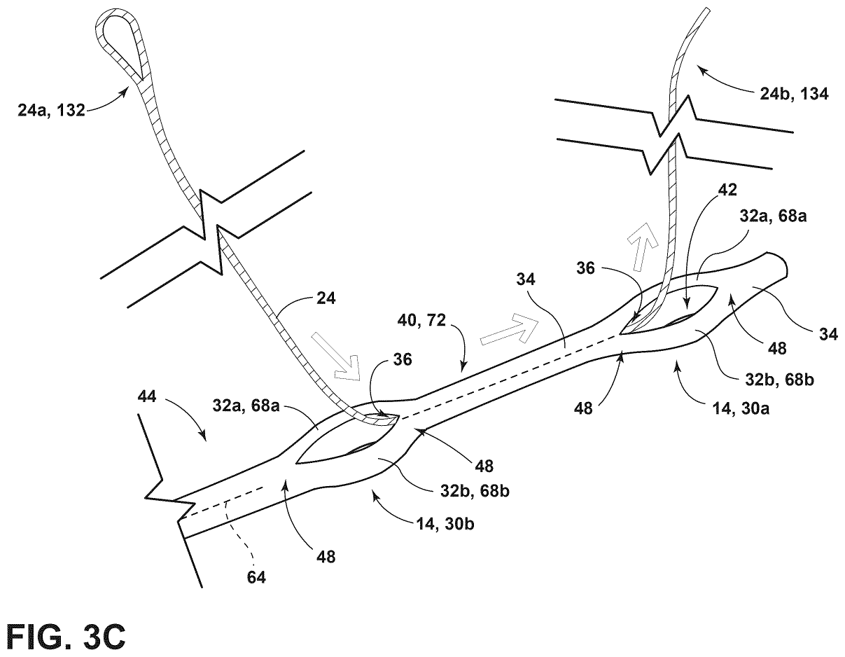

As described in further detail in reference to FIG. 3C, the access region 36 or opening in the cross-section of the suture 10 may facilitate the threading of shuttle suture 24 through the intertwined, continuous strands 12 between two consecutive windows 30 to define a splicing region 40. The shuttle suture 24 may be introduced centrally within the cross-section of the suture 10 via the access region 36 formed by a first window 30a, pass through the splicing region 40, and exit out through the access region 36 of a second window 30b. In this way, the shuttle suture 24 may be passed through the splicing region 40 to assist in threading the shuttle suture 24 into the suture 10. In various implementations, the shuttle suture 24 may be fed through the structure of the suture 10 formed by the continuous strands 12 or extend in parallel with the splicing region 40 and pass-through apertures 42 formed by the windows 30.

Referring now to FIGS. 2-5, the integrated features 14 of the suture 10 are described in further detail. As demonstrated in FIG. 2, a pictorial view of the suture 10 is shown generally demonstrating the integrated features 14 formed by the continuous strands 12 at a representative scale. The features 14 demonstrated in FIG. 2 are described with further structural detail and exemplary variations in FIGS. 3-5. As demonstrated in FIG. 2, the suture 10 may generally extend from a first end portion 10a to a second end portion 10b. In the example shown, the splicing region 40 may be positioned proximal to the first end portion 10a. Adjacent and proximal to the splicing region 40, an expanded or stuffed region 44 may extend along a neighboring portion of the continuous length L. Proximal to the second end portion 10b, a tapered region 46 may extend to a transition section 48 proximal to the stuffed region 44. In this configuration, the suture 10 may be fed through various structures or portions of surgical assembly 16 beginning with the tapered region 46 passing through one or more openings or eyelets, including the apertures 42 of the one or more windows 30. Further, the suture 10 may be secured or anchored in a knotless configuration based on the interaction between the expanded or stuffed region 44 engaging part of a surgical construct, for example, a lumen 52 or passage formed through the soft anchor 22, as previously demonstrated in FIG. 1.

In general, various features are described in the disclosure as being formed by automated manufacturing processes and particularly as being formed by the interlinked yarns or strands 12 along a continuous length L. However, it shall be understood that the continuous strands 12 may refer to the yarns that are braided, interwoven, or otherwise interlinked to form the specific features (e.g., bifurcated legs, transitions in cross-section, etc.) which may be associated with transition sections 48 as the continuous strands 12 formed by the automated processes disclosed. However, the resulting constructs or sutures including the underlying features may be formed from multiple constituent parts including additional stuffing or filling materials, spliced sections, combinations of sutures, wires, or various elements. Accordingly, the disclosure may provide for a wide variety of applications for several features that may be attributed to the automated or semi-automated manufacturing processes described.

Referring now to FIGS. 3A-3C, the integrated features 14 of the suture 10 are described in further detail. As shown in FIG. 3A, a detailed schematic view of the suture 10 is shown emphasizing the features demonstrated in FIG. 2. Further, FIG. 3B demonstrates a schematic diagram of the suture 10 as formed by an automatic braiding machine. For clarity, the configurations of the suture 10 demonstrated in FIGS. 3A and 3B may be referred to as a modified suture 60 and an unmodified suture 62. The modified suture 60 may refer to the trimmed or finished textile construct in FIG. 3A. The unmodified suture 62 may refer to the suture 10 as produced by an automated braiding machine demonstrated in FIG. 3B. In general, the process steps associated with the modified suture 60 relative to the unmodified suture 62 may be completed by one or more manual or machine-operated processes.

In general, each of the integrated features 14 of the suture 10 formed by the continuous strands 12 may be the result of one or more of the transitions 48 of the path intertwining the continuous strands 12. As demonstrated in FIG. 3B, each of the transitions 48 is demonstrated as a variation in the structure of the suture 10 varying from the continuous cross-section of the carrier braid 34. As discussed throughout the application, the cross-section and proportions of the carrier braid 34 may vary depending on the application. In general, the carrier braid 34 may correspond to a round, rectangular, and/or trapezoidal shape formed by the underlying braided or intertwined structure of the continuous strands 12. Each of the transitions 48 forming the integrated features 14 may result from variations in the path or paths and associated tracks of one or more horn gears and corresponding slots driving the bobbin carriers of the automated braiding equipment to dispense the continuous strands 12. For example, the windows 30 may be the result of a bifurcation of the continuous strands 12 forming the carrier braid 34. In this way, the apertures 42 formed by the windows 30 may be opened. The closure of the windows 30 may further be the result of a merger of the continuous strands 12 at an additional transition section 48. The merger of the continuous strands 12 may return the suture 10 to the structure of the carrier braid 34. For clarity, the carriers or bobbin carriers may refer to components of a braiding or textile manufacturing machine that may “carry” bobbins from which the yarns or strands 12 are dispensed. The carrier braid 34 described in reference to the suture 10 may correspond to a resulting interlinked structure formed by the strands 12 along the length of the suture 10.

Still referring to FIGS. 3A and 3B, the splicing region 40 may be the result of an over-braiding process completed by intertwining the continuous strands 12 of the carrier braid 34 over a stuffing suture 64. As shown in FIG. 3B, a first end 64a and second end 64b of the stuffing suture 64 may be embedded or enclosed by the continuous strands 12 forming the carrier braids 34 along a portion of the continuous length L. Though not demonstrated in FIG. 3B, in some implementations, the stuffing suture 64 may similarly be incorporated within the structure of the suture 10 by feeding or threading the stuffing suture 64 through one or more of the integrated features 14, similar to the threading of the shuttle suture 24 in the splicing region 40 as later discussed in reference to FIG. 3C. Accordingly, the disclosure may provide for the suture 10 to vary or increase in cross-section along the continuous length L by incorporating the stuffing suture 24 or various forms of film material to form the stuffed or expanded region 44.

The tapered region 46 demonstrated in FIG. 3A may result from another transition section 48 of the continuous strands 12 forming the suture 10. As shown, the continuous strands 12 may be bifurcated at the transition section 48, resulting in a plurality of tails 66, which may extend to the second end portion 10b of the suture 10. As shown, the plurality of tails 66 may include a first tail 66a and a second tail 66b. The bifurcation of the continuous strands 12 of the carrier braid 34 forming the tails 66 may be completed along one or more portions of the continuous length L, which may result in corresponding decreases in the cross-section of the suture 10 that may result from a churning process implemented to complete the modified suture 60.

As discussed herein, the carrier braid 34 or interlinked structure of the suture 10 may be formed by a plurality of intertwined or interlaced yarns, referred to as the continuous strands 12. The interlinked engagement among the yarns or strands 12 may be formed with a braiding machine having bobbin carriers configured to pass the strands 12 through one or more tracks around one or more axes (not shown). The braiding machine may have a first mode to form the non-bifurcated segments and a second mode to form the bifurcated segments or legs 32. In the first mode, the braiding machine may pass all of the strands 12 in a single path of travel, which may correspond to a common path or axis to form the non-bifurcated segments. In the second mode, the braiding machine may pass a first subset of the strands 12 in a first path or first axis to form the first leg 32a and a second subset of the strands 12 around a second path or axis of travel to form the second leg 32b. For example, the braiding machine may have a bar that passes between two different sets of carriers to transition the braiding machine from the first mode to the second mode. The bar may have channels that, when present, divides the single path of travel of the first mode into the two separate paths of the second mode to form the bifurcated legs 32.

Referring still to FIGS. 3A and 3B, the modified suture 60 may be manufactured from the unmodified suture 62 via simple processing steps that may be completed through limited automated or manual post-processing procedures. As demonstrated in FIG. 3B, the opposing first end 64a and second end 64b of the stuffing suture 64 may be trimmed from the carrier braid 34. Further, one of the tails 66, for example the second tail 66b, may be trimmed from the second end portion 10b of the suture 10. Compared to other possible manufacturing techniques, these steps may be completed with limited resources and time. Accordingly, the integrated features 14 of the suture 10 may limit process variation and improve manufacturability of the suture 10. In various implementations, the stuffing suture 64 may be formed from a filler material in the form of a suture. For example, stuffing suture 64 may have a diameter of 4-0 USP to 4 USP. For example, the diameter of the stuffing suture 64 may include a 4-0, 3-0, 2-0, 1-0, 0, 1, 2, 3, or 4 USP diameter. Accordingly, the stuffing suture 64 may be varied in diameter to adjust the proportions of the expanded or stuffed region 44.

Referring now to FIG. 3C, an exemplary procedure for feeding or threading the shuttle suture 24 through the splicing region 40 is shown. As previously discussed, the shuttle suture 24 may be fed through an access region 36 that may be exposed at the transition section 48 of the carrier braid 34 within the windows 30. Stated differently, the shuttle suture 24 or other sutures/filaments/materials introduced in the splicing region 40 may extend through a transverse opening formed by the access region 36 at the transition section 48. In this way, the shuttle suture 24 or other sutures, threads, or materials may be fed between the legs 32 of the window 30 and into a central portion 70 of the suture 10 extending into the carrier braid 34 via the aperture 42 formed between the legs 32. As further demonstrated in FIG. 3C, the shuttle suture 24 may extend through a bridge region 72 between the consecutive transitions 48 formed by the first and second windows 30a, 30b. In this way, the integrated features 14 of the suture 10 may provide access to thread the shuttle suture 24 through the bridge region 72 to readily and consistently extend the shuttle suture 24 through the tissue or connecting suture 10. Though previously discussed and described in various examples as a braided tissue or connecting suture, the suture 10 may correspond to various braided, woven, or intertwined textile constructs that may be implemented for a variety of surgical applications. In the context of the detailed description, the suture 10 is primarily described in reference to the tissue suture associated with the exemplary surgical assembly introduced in FIG. 1. However, it shall be understood that the corresponding structure of the suture 10 and the resulting integrated features 14 may be implemented in a variety of surgical and medical constructs without departing from the spirit of the disclosure.

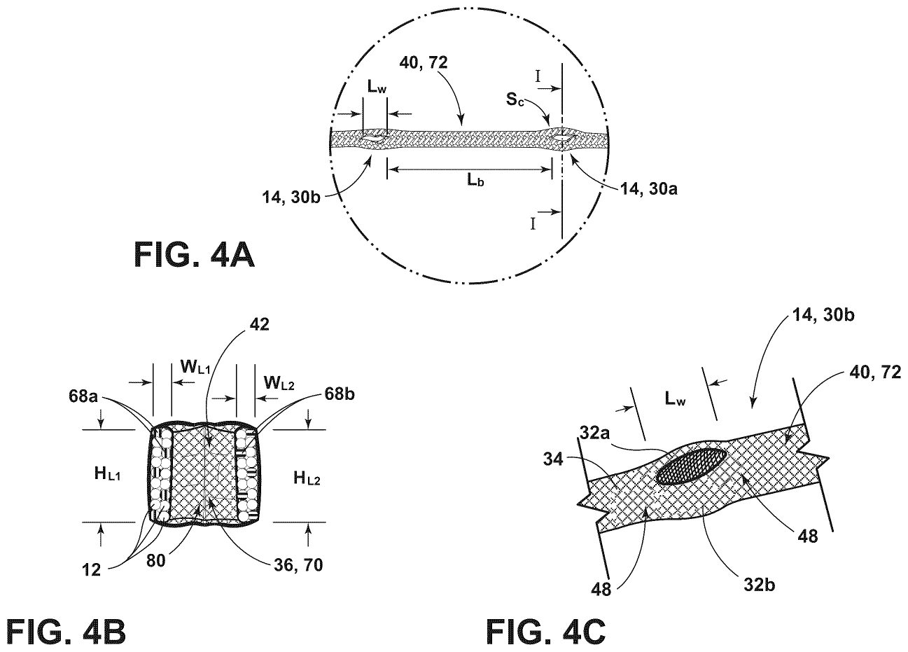

Referring now to FIGS. 3 and 4, one or more of the continuous strands 12 forming the legs 32 or tails 66 may have a color 68a, 68b controlled and adjusted along the continuous length L. For example, by adjusting the path of each of the yarns forming the continuous strands 12, the color of one or more of the continuous strands 12 forming the integrated features 14 may be controlled. In this way, the different legs 32, tails 66, or various features may be identified by one or more colors of the continuous strands 12 extending therethrough. In sections of the carrier braid 34 between the legs 32 and/or tails 66, the strands 12 of the first color 68a and the second color 68b may be intertwined. In this way, the strands 12 of the first color 68a may be separated from the strands 12 of the second color 68b where the legs 32 or tails 66 are bifurcated or joined over the transition sections 48. By forming portions of the features 14 in different colors, the resulting features 14 may be coded in different colors for ease of post-processing in manufacturing and/or identification for manipulation in various steps of a surgical procedure.

As best shown in FIG. 4B, the first leg 32a may include at least one of the strands 12 in the first color 68a. Additionally, the second leg 32b may include at least one of the strands 12 in the second color 68b. In this configuration, the sides of the suture 10 and the corresponding legs 32 may be readily distinguished based on the first color 68a and the second color 68b. The colors 68a, 68b associated with the continuous strands 12 forming the legs 32 may further extend to the plurality of tails 66. In this configuration, the first tail 66a may be identified based on the first color 68a and the second tail 66b may be identified based on the second color 68b. This configuration may be particularly beneficial for providing a visual indicator allowing the tails 66 to be distinguished based on the colors 68a, 68b for trimming as demonstrated in the trimming process in FIG. 3B.

Referring now to FIGS. 4A-4C, various configurations of the windows 30 are discussed in further detail. In general, the bridge region 72 extending between the integrated features 14, exemplified as the windows 30, may extend over a bridge length L_{B}. In various implementations, the bridge length may vary from approximately 0.5 mm to 128 mm. The legs 32 of the windows 30 may extend over a window length Lw, which may range from approximately 0.5 mm to 40 mm. Based on the proportions of the windows 30 and the intervening bridge length L_{B} of the bridge region 72, the splicing region 40 may be adjusted to suit or receive materials, for example, the shuttle suture 24, to accommodate a variety of surgical applications. Such variations in the lengths of the integrated features 14 of the suture 10 may be adjusted by changing or alternating the timing and corresponding lengths of the yarns forming the continuous strands 12 extending between the transition sections 48.

In addition to adjusting the proportions of the continuous length L between the transition sections 48 of the suture 10, the ratio of the number of strands 12 bifurcated between the legs 32 and/or the tails 66 may be varied. For example, in some cases, a strand density of the legs 32 forming the windows 30 or the tails 66 may vary from approximately 10 and 300 picks per inch (PPI). In addition to the density, the proportions of the legs 32 and/or tails 66 may also be adjusted based on the number of the continuous strands 12 bifurcated at the corresponding transitions 48. In this way, the corresponding height H_{L} and width W_{L} of the legs 32 may be adjusted to suit a particular application. Similarly, the height H_{T} and width W of the tails 66 also be adjusted based on the number of intertwined strands associated with each tail 66a, 66b bifurcated from the carrier braid 34. In this way, the proportions of the legs 32 and/or tails 66 may be adjusted to suit a particular application. Though discussed as the height and width of the bifurcated sections 32, 66, the proportions may be approximately equal forming the cross-sections of the legs and/or tails as round or square shapes.

As demonstrated in FIG. 4B, a cross-section of the legs 32 resulting from the bifurcation of the carrier braid 34 is shown demonstrating the central portion 70 extending into the carrier braid 34. In this example, the first leg 32a has a first height H_{L1} and a first width W_{L1}. Similarly, the second leg has a second height H_{L2} and a second width W_{L2}. In operation, the automated braiding process utilized to form the suture 10 may adjust a strand count associated with each of the legs 32 to be equal, as exemplified in FIG. 4B, or vary. For example, the count of the continuous strands 12 forming the carrier braid 34 may range from approximately 8 carriers or strands to 56 carriers or strands, or even more in some cases. Accordingly, the path along which the strands 12 are dispensed by the carriers may be adjusted to vary or alter the split associated with the bifurcation that occurs at each of the transitions 48 producing the windows 30. Similarly, the ratio of the strands 12 may also be split at the bifurcation forming the transition 48 of the tails 66. In this way, the proportions of each of the legs 32 or tails 66 forming the integrated features 14 of the suture 10 may be varied based on the path of the carriers driving the corresponding strands 12 in the automated braiding process.

As previously discussed, carriers and associated strands 12 forming the carrier braid 34 may range from approximately 8 to 56 carriers or more. For example, the continuous strands 12 forming the suture 10 may be dispensed from 12 carriers, 16 carriers, 20 carriers, 24 carriers, 28 carriers, 32 carriers, 40 carriers, 48 carriers, 56 carriers, or more. Depending, in part, on the number of carriers, the ratio defining the proportions (e.g., leg height H_{L}, leg width W_{L}, and strand count S_{C} of each of the legs 32 or tails 66) may vary based on a ratio of the split among the continuous stands 12 formed at the transition 48. In various implementations, the ratio of the strands 12 between the legs 32 or tails 66 may be even at a 1:1 ratio. However, in some implementations, the legs 32 or tails 66 may have differing numbers of the continuous strands 12 forming a ratio between the underlying strand counts S_{C}. In some implementations, the ratio between the legs 32 and/or tails 66 may be 5:3 or greater. Some examples of ratios between the strands 12 forming the legs 32 or tails 66 may be 2:1, 3:1, 9:1, etc. In various implementations, the ratio between the continuous strands 12 forming the legs 32 or tails 66 may vary in correspondence with the desired proportions of the leg height H_{L} and leg width W_{L} or a corresponding tail height H_{T} or tail width W_{T} of the strands 12 forming the legs 32 or tails 66 of the suture 10. In this way, the cross-sectional proportions of the legs 32 and/or the tails 66 may be varied to implement the integrated features 14 of the suture 10 for a variety of applications. As previously described, carriers associated with the manufacturing operations disclosed may “carry” bobbins from which the yarns or strands 12 are dispensed. Accordingly, the carrier count of the resulting carrier braid 34 may refer to the number of strands 12 dispensed from the bobbins by the carriers. The term “carriers” may relate to nomenclature associated with a textile manufacturing process (e.g., braiding, weaving, etc.) related to those disclosed. However, the terms and related description are not limited to the associated manufacturing processes unless specifically claimed.

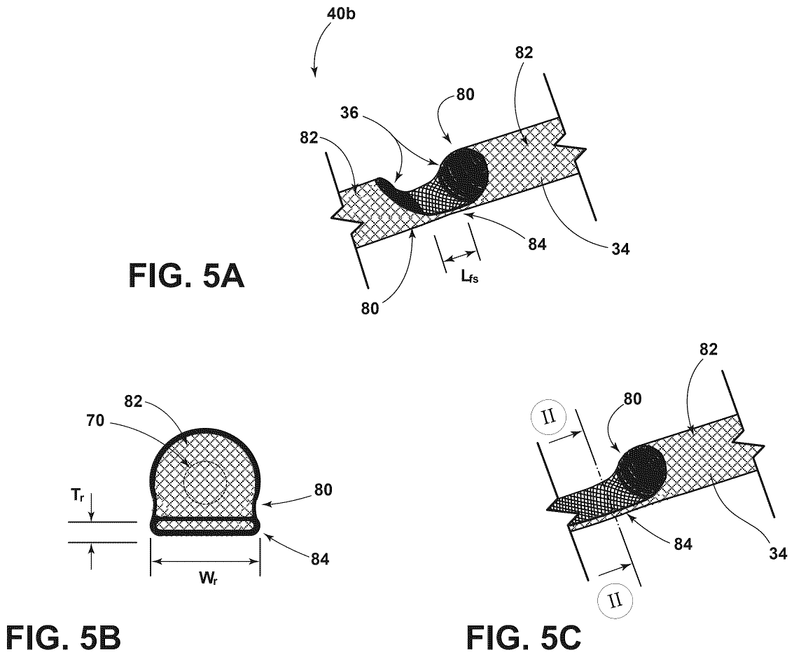

Referring now to FIGS. 5A-5C, additional examples of the integrated features 14 formed from the continuous strands are shown in the form of a cross-sectional transition section 80. In the specific example shown, the cross-sectional transition 80 is demonstrated in the form of a transition from a round cross-section 82 of the carrier braid 34 to a flat cross-section 84. In various implementations, forming the transition section 48 as the cross-sectional transition section 80 may similarly provide access to the central portion 70 of the carrier braid 34 as discussed in reference to the windows 30. In the example shown, the cross-sectional transition section 80 may extend over a transition point along which the central portion 70 of the continuous stands 12 forming the carrier braid 34 may be exposed. In this way, the central portion 70 may be accessible to thread or feed one or more sutures or filler materials through the continuous length of the suture 10.

As shown in the example of FIG. 5A, opposing transition sections 48 may adjust the cross-section of the suture 10 from a round cross-section 82 to a flat cross-section 84 back to the round cross-section 82. In such implementations, the flat cross-section 84 may form a flat suture length LFs extending between the neighboring portions of the suture 10 having the round cross-section 82. Additionally, successive instances of the cross-sectional transition section 80 may be implemented in combination to form a bridge region 72 similar to the windows 30. For example, the cross-sectional transition from the flat cross-section 84 of the round cross-section 82 may be followed along the continuous length L of the suture 10 by one or more additional cross-sectional transition sections 80 or windows 30, which may expose the central portion 70 of the carrier braid 34. In this way, the cross-sectional transition sections 80 and/or the windows 30 or other transition sections 48 may be implemented in various combinations to provide bridge regions or sections between the integrated features 14, which may define splicing regions.

As demonstrated in FIG. 5C, the flat cross-section 84 may extend the cross-sectional transition section 80 to a distal end of the suture 10 and/or be bifurcated into two or more flattened tails. Accordingly, the cross-sectional transition section 80 and resulting round and flat cross-sections 82, 84 of the suture 10 may be varied in length to suit a variety of applications. In various implementations, the flat cross-section 84 may be implemented similar to the tapered region 46, allowing the suture 10 to pass through narrow passages. Additionally, the flat cross-section 84 may assist in controlling a bending direction of the continuous length L of the suture. For example, the suture 10 may readily bend along a ribbon thickness T_{R} while limiting bending over a ribbon width W_{R}. In this way, the suture 10 may be configured to provide improved control and manipulation for various procedures.

As demonstrated in FIG. 5B, the central portion 70 of the carrier braid 34 is shown extending through the cross-sectional transitional section 80 and into the round cross-section 82. In various implementations, the proportions of the round cross-section 82 and flat cross-section 84 may be adjusted, primarily based on the strand count Sc and corresponding carriers used to form the suture 10. Additionally, the proportions of the flat cross-section 84, including the ribbon thickness T_{R} and the ribbon width W_{R}, may be varied based on the arrangement of the intertwined continuous strands 12 forming the suture 10. For example, the number of carriers may range from approximately 8 to 56 or more and the corresponding strands may be arranged in various proportions or ratios to adjust a ribbon thickness T_{R} relative to the ribbon width W_{R}. In this way, the proportions of the flat cross-section 84 or modified rectangular cross-section may be adjusted to expose more or less of an interior of the round cross-section 82 and corresponding extent of the central portion 70 accessible along the continuous length L of the suture 10.

Though described as flat sections 84 and round sections 82, the cross-section of the suture 10 and the corresponding transition sections 80 may correspond to various shapes including rectangular, square, ovular, or additional cross-sectional shapes that may be formed by the continuous strands 12. Accordingly, the terms round and flat may be referenced for simplicity, particularly in reference to the associated figures. However, for the purposes of the disclosure, the term flat may correspond to various flattened shapes that may correspond to ovals, rectangles, or other oblong cross-sections. Alternatively, the round cross-section may generally correspond to or provide for a variety of shapes that may be similar cross-sectional proportions (e.g., height and width) that may be round, square, rectangular, tubular, or form various shapes. Accordingly, the cross-sectional transition sections 80 may be formed by the interlinked continuous strands 12 or yarns to form various shapes that may be adjusted along the length L of the suture 10 responsive to the transition sections resulting from changes in the paths of the interlinked yarns forming the strands 12.

Referring now to FIGS. 6A-6E, an exemplary implementation of the suture 10, as previously introduced in reference to FIG. 1, is described in reference to a clinical application or a tissue repair construct 130. Referring first to FIG. 6A, the surgical assembly 16 previously referenced in FIG. 1 is demonstrated in an assembled configuration. As shown, the stuffing suture 64 or filler material extends through a portion of the suture 10 proximal to the second end portion 10b. The second end portion 10b is formed by the tapered region 46 and may be threaded through the aperture 42 forming the first window 30a to form the surgical assembly as shown. Opposite the second end portion 10b, the first end portion 10a may include the first window 30a and extend along the splicing region 40 through the lumen 52 formed by the soft anchor 22. In this configuration, the suture 10 may extend from the first end 10a through the soft anchor 22 and loop on itself from the tapered region 46 through the first window 30a as shown. Additionally shown in FIG. 6A, the shuttle suture 24 may extend through the lumen 52 in the soft anchor 22 within the splicing region 40 between the second window 30b and the first window 30a. As further shown, a first end portion 24a of the shuttle suture 24 may form a passing loop 132. Opposite the first end 24a, a second end 24b of the shuttle suture 24 may form an interface end 134 or pulling end that may be utilized to pass the tapered region 46 around patient tissue 136 or an object to secure the patient tissue 136 in a tensionable loop 138 without requiring a knot or other tensioning devices.

As shown in FIG. 6C, the soft anchor 22 or, more generally, anchor construct 18 may be positioned within a pocket 140 that may be formed in a bone or support structure 142 of a patient. Following the deposition of the anchor construct 18 and corresponding portions of the suture 10 and shuttle suture 24 within the pocket 140, the tapered region 46 at the second end portion 10b of the suture 10 may be threaded through the passing loop 132, as shown in FIG. 6B. Once the tapered region 46 is looped about and secured to the passing loop 132, the surgeon may apply tension to the interface end 134 of the shuttle suture 24 and begin to feed the second end portion 10b of the suture 10 back into the soft anchor 22 and further into and through the splicing region 40 formed between the windows 30.

The result of the passage of the suture 10 through the splicing region 40 is shown in FIG. 6D. As shown, the shuttle suture 24 is extended out from the pocket 140 and has drawn the tapered region 46 of the suture 10 through the splicing region 40. In this configuration, the surgeon may apply further tension to the second end portion 10b of the suture 10, thereby securing the patient tissue 136 to the tissue or construct within which the pocket 140 is formed in an anchor tissue or anchor material to retain the patient tissue 136. In this way, the suture 10, including the integrated features 14, may be implemented to provide the tissue repair construct 130 via an improved process that may limit variation and improve manufacturing ability. In doing so, the suture 10, in this case the tissue suture, may improve patient outcomes associated with a variety of surgical procedures.

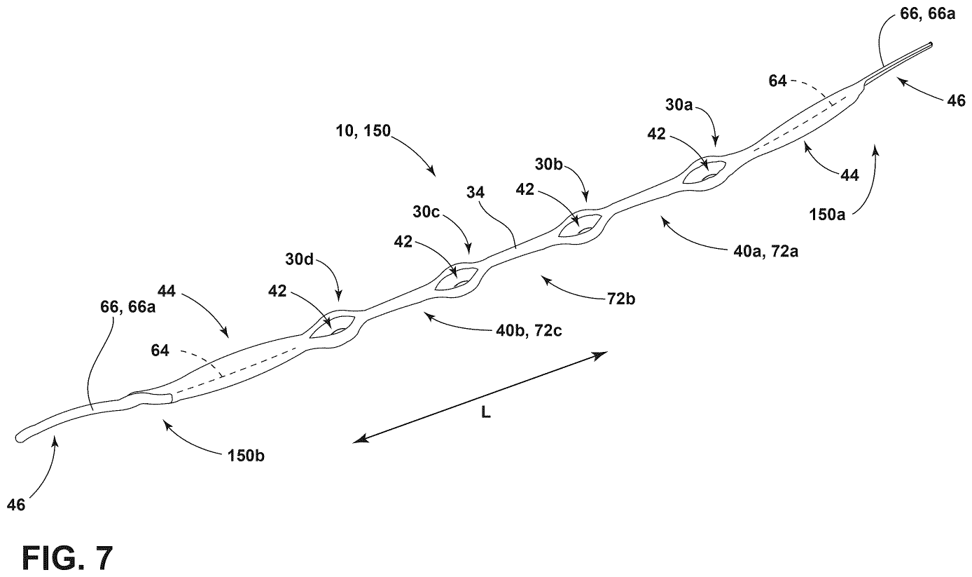

Referring now to FIG. 7, the suture 10 (e.g., a tissue suture) is shown demonstrating an extended symmetric implementation 150 including similar integrated features 14 as previously discussed that may be manufactured from automated textile manufacturing processes (e.g., braided, woven, etc.). In the example shown both a first end portion 150a and a second end portion 150b of the suture 10 include the tapered region 46 and the expended or stuffed region 44. Additionally, the plurality of windows 30 may include a first window 30a, a second window 30b, a third window 30c, and a fourth window 30d. In this configuration, the suture 10 may include a plurality of bridge regions 72. More specifically, the suture 10 may include a first bridge region 72a, a second bridge region 72b, and a third bridge region 72c. One or more of the bridge regions 72 may define splicing regions 40 between the windows 30. In the example shown, the suture 10 includes a first splicing region 40a and a second splicing region 40b separated by section of carrier braid 34 about which the integrated features 14 may be symmetrically formed. In this way, the suture 10 may provide for multiple tensionable loops 138 that may be in connection with a common anchor construct 18 or multiple, neighboring anchors. Accordingly, the disclosure may provide for a variety of arrangements of the features 14 to be formed along the continuous length L by the plurality of continuous strands 12 or intertwined yarns.

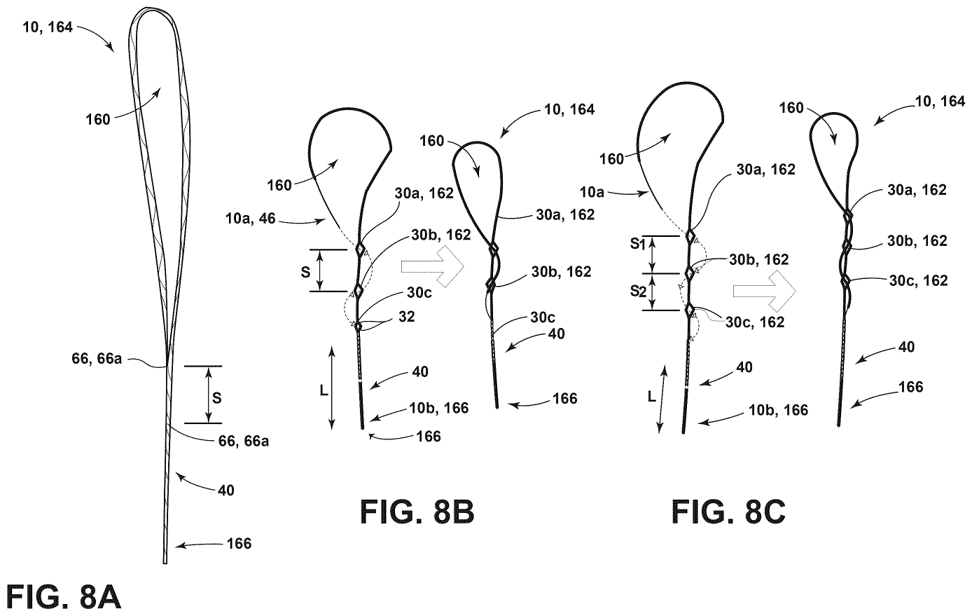

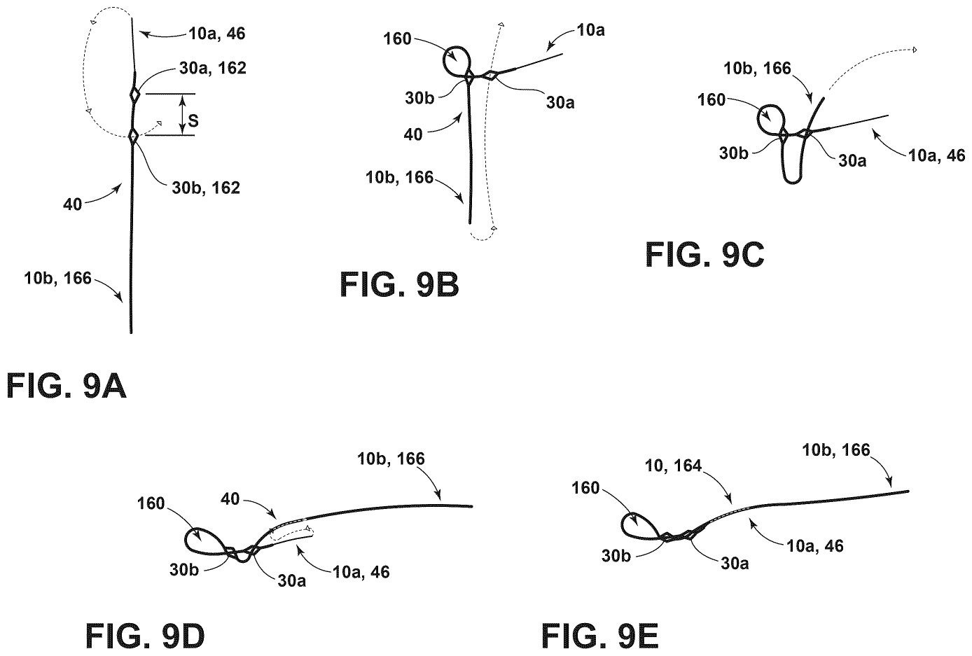

Referring now to FIGS. 8 and 9, additional configurations of the suture 10 are shown demonstrating various suture loops 160 formed by splicing sections of the suture 10 along the length. As previously disclosed, the suture 10 may include a plurality of the windows 30 formed by the interlaced continuous strands 12 transitioning from the combined carrier 34 or carrier braid section to the bifurcated legs 32 and back to the combined carrier section 34. Without the windows 30 formed along the length L, the placement of pierce points 162 or passing point, where the length L of the suture 10 is threaded through its own cross-section, may be manually positioned along the length. Such manual positioning steps may be based on measurements and/or marking steps designating the positions of the pierce points 162. These steps are time-consuming and create potential for errors in measurement and placement of the pierce points 162. Further, a manual piercing process may cause variations in resulting spliced suture constructs 164 that may result from inconsistent threading of the suture 10 through the strands 12 forming the cross-section. As demonstrated in the examples presented in FIGS. 7 and 8, the position of the windows 30 along the length L may ensure that a spacing S between two or more pierce points 162 is controlled based on a placement of the windows 30 along the length L. In this way, the spacing S of the pierce points 162 used to form the suture loop 160 may be controlled based on an automated manufacturing process associated with the manufacture of the suture 10.

As best demonstrated in FIG. 8A, an example of the spliced suture construct 164 is shown demonstrating the relative scale and proportions. The suture 10 utilized to form the suture loop 160 includes two of the windows 30a, 30b separated over a predetermined spacing S, as shown in further detail in FIG. 8B. Additionally, in some implementations, the suture 10 may further include a tapered region 46 formed at the first end 10a proximal to the first window 30a. In general, the loop 160 may be formed by feeding the first end 10a through the first window 30a and the second window 30b successively along the length L of the suture 10. The location where the end 10a of the suture 10 passes through its cross-section formed by the strands may correspond to the pierce points 162. As shown in FIG. 8B, the windows 30 designating the pierce points 162 may be readily identified based on the separation of the legs 32a, 32b forming the apertures 42 of the windows 30. In this configuration, the windows 30 may help ensure that the resulting spliced suture constructs 164 are manufactured with accurate spacing S and positioning of the pierce points 162 along the length.

Still referring to FIG. 8B, following the passage of the first end portion 10a of the suture 10 through the second window 30b, the first end portion 10a may further be spliced or tucked into the carrier braid along a splicing region 40. As previously disclosed, the splicing region 40 may also be defined along the length L of the suture by one of the transition sections 48, which may correspond to the legs 32 formed by the bifurcation of the carrier 34 or the cross-sectional transition section 80. In either case, the transition sections 48 may form the access region 36 into the cross-section of the carrier braid 34 which may improve the ease of threading the first end 10a of the suture 10 into the splicing region 40 and the quality of the resulting spliced suture construct 164. Additionally, as previously discussed, the proportions of the bifurcated legs 32 forming the windows 30 may vary in proportions (e.g., strands or yarns) as well as window length Lw to further improve the structure of the suture 10. Accordingly, the disclosure may provide for transitions sections 48 formed along the length of the suture 10 to provide a wide range of beneficial structures to suit correspondingly diverse surgical applications.

Referring now to FIG. 8C, another example of the spliced suture construct 164 is shown demonstrating a first window 30a, a second window 30b, and a third window 30c. As shown, the number of windows 30 may vary depending on the application. Additionally, the spacing S among the windows 30 may vary. As shown, the first window 30a and the second window 30b are separated over a first spacing S₁, and the second window 30b and the third window 30c are separated over a second spacing S₂. As shown, the first spacing S₁ and the second spacing S₂ may differ. For example, the first spacing S₁ may be greater than the second spacing S₂. In this way, the positions of the pierce points 162 may be placed and planned based on the positions and the spacing S of the windows 30 along the length L.

Referring now to FIGS. 9A-9E, another example of the suture 10 is shown demonstrating a variation of a manufacturing method for forming the suture loop 160 from the spliced suture construct 164. Due to the complexity of the threading or manufacturing process, sequential steps A-E are shown demonstrating how the suture construct 164 is formed. As shown in step A, the structure of the suture 10 is similar to that of FIG. 8B. However, steps B and C demonstrate that the loop 160 may be formed from different splicing or piercing steps. As shown in step B, the first end portion 10a is threaded through the second window 30b. In step C, the second end portion 10b of the suture 10 is then passed through the first window 30a, which is shown protruding through the second window 30b in step B. In step D, the second end portion 10b is drawn through the first window 30a, such that an elongated tail 166 is formed generally opposite the loop 160 formed by the suture construct 164. The first end portion 10a is then spliced with the elongated 166 proximate to the second window 30b. In this way, the spliced suture construct 164 may be implemented to form the suture loop 160 in another exemplary configuration.

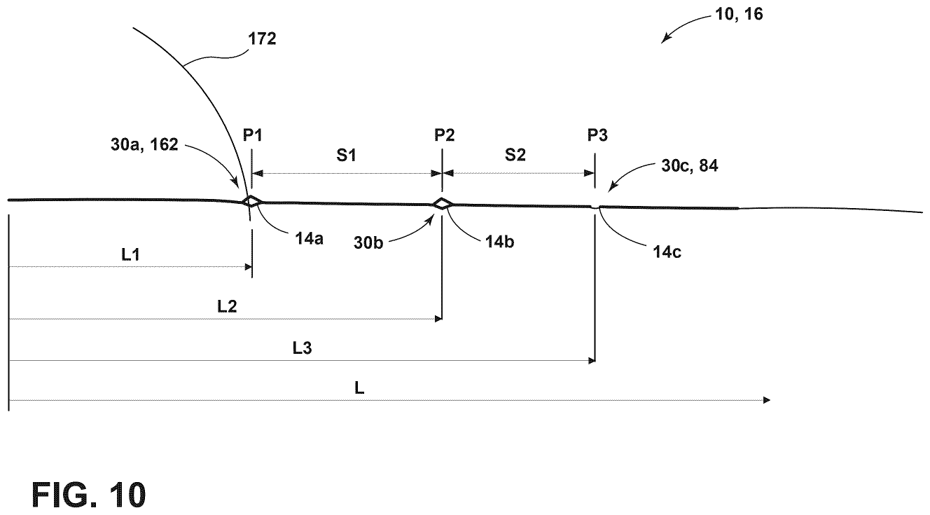

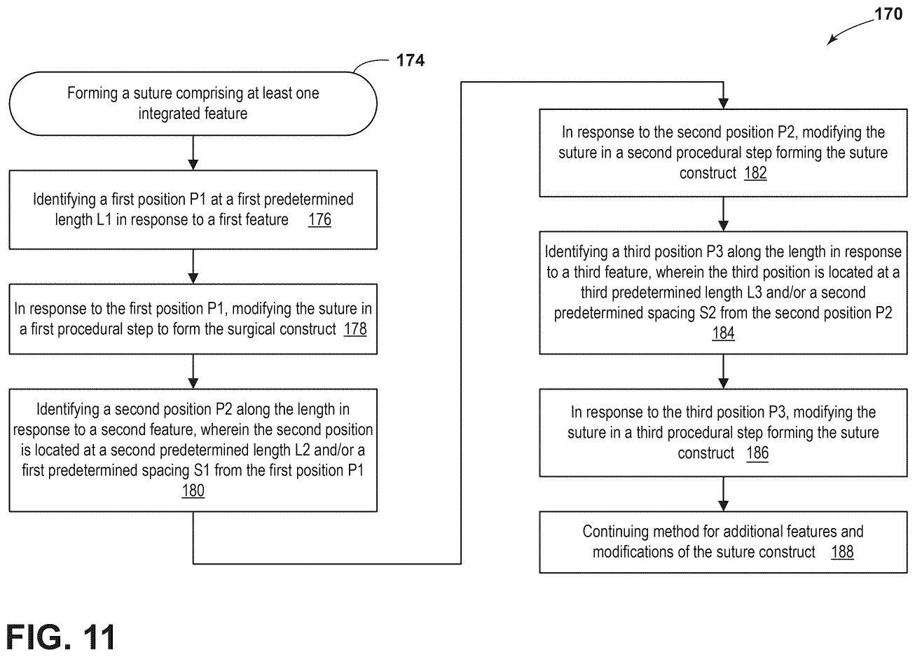

Referring now to FIGS. 10 and 11, a method 170 forming a suture construct or surgical assembly 16 is described in reference to the exemplary suture 10. In the example shown, the suture 10 includes the integrated features 14 located at a plurality of predetermined positions P1, P2, P3 along the length L. The predetermined positions P1, P2, P3 may be defined as predetermined lengths L1, L2, L3 and/or predetermined spacings S1, S2 that may be designated and identified based on the positions of the integrated features 14 along the length L. As further discussed in reference to the method 170, the position of one or more of the integrated features 14 may provide both integrated visual indications of the corresponding predetermined position and/or spacing along the length L. Additionally, the integrated features 14 may assist in one or more modifications to the suture 10 (e.g., splicing, stuffing, piercing, etc.). As previously discussed, the modifications to the suture 10 may result in the formation of various surgical assemblies 16 that may be implemented by engaging a portion of the suture 10 within itself and/or with one or more modification structures or assembly components 172 (e.g., sutures, filling materials, cores, needles, etc. Though demonstrated in FIG. 10 in reference to the passage of the assembly component 172 through the window 30, it shall be understood that the positions P1, P2, P3 of one or more of the integrated features 14 may assist in both the identification of the corresponding length L1, L2, L3 and/or spacing S1, S2 along the length L of the suture 10 to facilitate or assist in various passing modifications, bridging modifications (e.g., see FIG. 3C), splicing modifications, and various modifications that may be applied to the length L of the suture 10. In this way, the method 170 may provide for the identification of one or more positions along the length L of the suture 10 based on the integrated features 14 to assist in dimensional positioning of various modifications and further assist in completing the corresponding procedures associated with the modifications (e.g., piercing, passing, bridging, splicing, stuffing, tying, trimming, etc.).

As demonstrated in the flow chart of FIG. 11, the method 170 may begin in step 174 by forming the suture 10 including one or more of the integrated features 14. As previously discussed, the formation of the suture 10 with the integrated features 14 may be the result of one or more automated or semi-automated processes to intertwine the continuous strands 12 along the length L. Based on the associated programming and spacing of each of the underlying strands or yarns forming the suture 10, the resulting features 14 may be consistently located at corresponding dimensional positions P1, P2, P3 positioned at predetermined lengths L1, L2, L3 and/or spacings S1, S2, etc. Accordingly, in step 176, the first position P1 may be identified at the first predetermined length L1 in response to the position of a first feature 14a (176). With the first position P1 identified, the method 170 may continue by modifying the suture in a first procedural step to form the surgical assembly 16 or construct (178).

As previously discussed, the modifications to the suture 10 may include various manual or semi-automated procedures that may involve manipulating the suture 10, the assembly component 172, and/or various secondary assemblies (e.g., anchors, stuffing, sutures, connectors, pledgets, etc.) to form the surgical assembly 16. In this way, the positioning of the at least one integrated feature 14a at the first position P1 based on the position of the first feature 14a may ensure the corresponding feature of the surgical assembly is accurately positioned without requiring additional measurement or process control operations. Accordingly, the predetermined positions P1, P2, P3 of the integrated features 14 may improve the dimensional accuracy of the formation of the surgical assembly 16 and limit the time associated with measuring or verifying the corresponding positions for the modification along the length L. Though discussed in reference to a singular modification associated with the integrated features 14, it shall be understood that each of the integrated features 14 may be implemented in one or more consecutive or disparate steps to accomplish the assembly method 170.

Still referring to FIG. 11, following the modification of the suture in the first procedural step, the method 170 may continue by identifying a second position P2 along the length L in response to a second feature 14b of the suture 10 (180). As previously discussed, the second position P2 may be located at a second predetermined length L2 and/or first predetermined spacing S1 from the first position P1. Additionally, the second position P2 of the second feature 14b may be located at a second predetermined spacing S2 from a third position P3 of a third feature 14c. Accordingly, in response to identifying the location of the second position P2 based on the second feature 14b, the suture 10 may be modified in a second procedural step, thereby forming the surgical assembly 16 (182). Following the second procedural step, the method 170 may continue by identifying a third position P3 along the length L in response to the third feature 14c (184). Similar to the first position P1 and the second position P2, the third position P3 may be located at a third predetermined length L3 and/or second predetermined spacing S2 relative to the second position P2. In response to identifying the location of the third position P3 based on the third feature 14c, the method 170 can continue by modifying the suture 10 in a third procedural step further forming the surgical assembly 16 or construct (186).

Following step 186, the method 170 may continue to modify the suture 10 by manipulating the suture and/or various components of the surgical assembly 16 that may be accurately located along the length L based on the predetermined positions P1, P2, P3 of the integrated features 14 (188). As previously discussed in various examples, the modifications and procedural steps applied to form the surgical assembly 16 from the suture 10 may be facilitated and/or assisted based on the beneficial structures formed by the transition sections 48 including the formation of the windows 30, splicing regions 40, and/or access regions 36 formed by the integrated features 14. In this way, the disclosure may provide for improved manufacturing and processing techniques to manufacture surgical assemblies 16 including various features that may be provided economically and with a high level of dimensional accuracy. In general, each of the predetermined positions P1, P2, P3 of the integrated features 14 may be positioned with a high level of accuracy based on the repeatability of the corresponding intertwining or interlacing processes for manufacturing the suture 10 via the automated or semi-automated process (e.g., a braiding or weaving process).

The materials forming the textile construct may correspond to continuous or semi-continuous strands or filaments. In general, the strands or filaments may be formed from natural or synthetic materials depending on the application. Examples of natural materials include catgut, silk, and cotton. Synthetic materials include nylon, polypropylene, polybutester, and stainless steel. Further, some materials associated with the strands or filaments of the textiles or structures disclosed include absorbable materials and non-absorbable materials. Examples of absorbable materials include PDS (polydioxanone), Vicryl (glycolide and lactide), polyglactin 910, polyglycolic acid, poliglecaprone, polyglycolide-trimethylene carbonate, polyglycone 6211, and glycomer 631 are examples of absorbable sutures. Non-absorbable materials include silk, polybutester, braided or intertwined polyester, nylon, polypropylene, polyhexafuropropylene, goretex, and stainless steel. In some implementations, one or more of the strands or filaments may be formed from shape memory materials (SMMs), which are materials that can return to their original shape after being deformed. Shape memory materials may include shape memory alloys or polymers including nitinol, Austenite, copper-aluminum-nickel, copper-zinc-aluminum, and iron-manganese-silico, polytetrafluoroethylene (PFTE), polylactide (PLA), and ethylene-vinyl acetate (EVA). Accordingly, the constructs discussed may be manufactured from a variety of materials.

According to some aspects of the disclosure, a suture formed from a plurality of continuous strands includes a continuous length of a carrier braid extending from a first end portion to a second end portion and a plurality of windows. The windows including a plurality of legs bifurcated from the continuous strands and defining at least one window length.

According to various aspects, the disclosure may implement one or more of the following features or configurations in various combinations:
the windows are separated by a bridge length of a window bridge of the suture;
the bridge length of the window bridge is between 0.5 mm and 128 mm;
the bridge length defines a splicing region of the carrier braid configured to receive and pass a length of suture;
the plurality of windows comprises three or more windows separated by a plurality of window bridges extending along the length;
the windows comprise a first window extending over a first window length and a second window extending over a second window length, wherein the first window length is greater than the second window length;
the at least one window length of the windows is between 0.5 mm to 40 mm;
a strand density of the windows is between 10 and 300 picks per inch (PPI);
the continuous length of the carrier braid comprises a cross-section of a round shape, a rectangular shape, or a flattened/ribbon-like shape;
the continuous strands forming the round shape or the rectangular shape form sidewalls that are bifurcated, forming the legs of the windows;
the windows each form an opening to a central portion of the cross-section of the carrier braid;
the openings in the central portion are configured to receive and pass a shuttle suture through a window bridge extending over a bridge length between the plurality of windows;
the legs forming at least one of the windows comprise an equal number of the continuous strands;
a first strand of the continuous strands forming the first leg is a first color, and a second strand of the continuous strands forming the second leg is a second color, different from the first color;
the legs comprise a first leg and a second leg, wherein the first leg comprises more of the continuous strands than the second leg;
a ratio of the continuous strands forming the first leg relative to the second leg is 5:3 or greater;
a split section includes a first tail and a second tail formed from the continuous strands of the carrier braid at the second end portion;
a first strand of the continuous strands forming the first tail is a first color, and a second strand of the continuous strands forming the second tail is a second color, different from the first color;
a ratio of the continuous strands forming the second tail relative to the first tail is 5:3 or greater (e.g., 5:3, 2:1, 3:1, 5:1, 9:1);
the second tail is trimmed from the continuous strands and the first tail of the split section forms the second end portion as a tapered end;
the windows comprise a first leg and a second leg forming opposing sides of the windows and wherein the first tail portion is formed from the continuous strands forming the first leg and the second tail is formed from the continuous strands forming the second leg;
the plurality of windows comprise a first window and a second window separated by a window bridge;
the plurality of windows further comprise a filled section positioned between the split section and the second window;
the filled section and the split section are mirrored on opposing sides of the plurality of windows forming the first end portion and the second end portion;
the first end portion is formed by the carrier braid adjacent the first window;
the carrier braid is formed by between 8 and 56 carriers, for example, 12 carriers, 16 carriers, 20 carriers, 24 carriers, 28 carriers, 32 carriers, 40 carriers, 48 carriers, or 56 carriers;
an expanded section formed along a stuffed portion of the continuous length of the suture;
the expanded section is formed by a filler material extending within the carrier braid;
the expanded section is formed by braiding the carrier braid over a filler material;
the filler material has a diameter of 4-0 USP to 4 USP (e.g., 4-0, 3-0, 2-0, 1-0, 0, 1, 2, 3, 4 USP);
the expanded section is positioned outside the windows along the continuous length;
the plurality of windows comprises at least one fill window positioned on a first side of the expanded section;
a filler material forming the expanded section is fed into a central portion of an intertwined cross-section of the suture through the at least one fill window;
the at least one fill window includes a first window positioned on a first side of the expanded section and a second window positioned on a second side of the expanded section, wherein the filler material is fed into the expanded section between the first window and the second window;
at least one of the plurality of windows is implemented interchangeably with a transition section of the carrier braid transitioning from a round or rectangular cross-section to a flat or ribbon-like cross-section, wherein the transition forms an opening to a central portion of the cross-section of the carrier braid;
a shuttle suture extending through a bridge length of a window bridge between the plurality of windows;
a fixation device engaged by the suture and configured to directionally engage a hole or pocket formed in a patient anatomy to retain the suture;
the fixation device comprises a soft anchor or a threaded anchor;
the soft anchor comprises a sheath forming a cannulation, the cannulation configured to pass through at least a portion of the continuous length and bind on the expanded section;
tension is applied to the shuttle suture and, in response to the tension, the tapered end of the suture passes through the opening formed by the second window and is passed through the splice region over the bridge length; and/or
as the continuous length of the suture passes through the sheath, the expanded section engages the second window and binds on the second window and the sheath, and in response to the binding, the shuttle suture is slidably withdrawn and released from the splicing region of the suture and the sheath.

According to another aspect of the disclosure, a method for providing a suture with an automated braiding machine is shown. The method includes braiding a carrier suture from a length of continuous strands extending from a first end portion to a second end portion and bifurcating the continuous strands along a pair of legs defining a plurality of windows including a plurality of legs from the continuous strands at least one window length.

According to various aspects, the disclosure may implement one or more of the following features or configurations in various combinations:
defining the plurality of windows includes joining the legs between the plurality of windows forming a window bridge over a bridge length;
the legs of the windows are formed from sidewalls of the carrier braid forming a round shape or a rectangular shape form;
bifurcating the continuous strands along the pair of legs exposes an opening in a central portion of a cross-section of the carrier braid;
the opening is exposed between the sidewalls of the carrier braid;
the plurality of windows comprises three or more windows separated by the plurality of window bridges extending along the length;
bifurcating the continuous strands of the carrier suture into a split section including a first tail and a second tail forming the second end portion;
the continuous strands forming the first tail comprise a first strand of a first color and a second strand of a second color, different from the first color;
forming an expanded section from the continuous length between the second window and a split portion forming the second portion;
the plurality of windows comprise a first window and a second window separated by a window bridge;
a filled section may be positioned between the split section and the second window;
the expanded section is formed by braiding the carrier braid over a filler material;
forming a fill window of the plurality of windows, wherein the fill window comprises at least one fill opening positioned on a first side of the expanded section;
feeding the filler material into the carrier suture through the at least one fill window, the fill window forming the expanded section;
feeding a shuttle suture through the splicing region through the opening in the central portion between the plurality of windows;
feeding the suture through an opening, an eyelet, a grommet, or sleeve of a fixation device;
the fixation device includes an anchor configured to directionally engage a hole or pocket formed in a patient anatomy to retain the suture;
trimming the second tail forming of the split section forming a tapered end of the second end portion; and/or
the second tail is distinguished from the first tail and trimmed based on the second color indicating the second tail is to be removed.

According to some aspects of the disclosure, a suture formed from a plurality of continuous strands including a continuous length of a carrier braid extending from a first end portion to a second end portion and a plurality of transition sections of the carrier braid from a round cross-section to a flat or ribbon-like cross-section. The plurality of transition sections comprise opposing, adjacent round to flat transitions that form an opening to a central portion of the cross-section of the carrier braid. The plurality of transition sections along the continuous length define a splicing region over a splice length between the transition sections forming the grouped transition. The splicing region is configured to pass a suture or insert between the transition sections.

According to various aspects, the disclosure may implement one or more of the following features or configurations in various combinations:
the at least one transition section includes opposing, adjacent round or rectangular to flat transitions or a grouped transition;
the adjacent round or rectangular to flat transitions form a pocket in the carrier braid defined on opposing longitudinal ends by the transition sections;
consecutive instances of the grouped transition along the continuous length define a splicing region over a splice length between the transition sections forming the grouped transition;
the splicing region is configured to receive a splicing needle and pass a shuttling suture;
the at least one transition section comprises a plurality of transition sections separated over the continuous length over a bridge length;
the at least one transition section further comprises a window section bifurcated from the plurality of continuous strands along a first leg and a parallel second leg; and/or
a strand density of the at least one transition section is between 10 and 300 picks per inch (PPI).

According to another aspect of the disclosure, a method for forming a surgical construct includes providing a suture comprising at least one integrated feature; identifying a first position of a first predetermined length of the suture; and in response to identifying the first position, completing at least one modification of the suture at the first position forming the surgical construct by interfacing with the at least one integrated feature.

According to various aspects, the disclosure may implement one or more of the following features or configurations in various combinations:
the at least one integrated feature is formed by a plurality of continuous strands that form the suture;
a dimensional accuracy of a position of the first modification is designated by the first position of the first feature;
stuffing an assembly component into a central portion of the suture;
the central portion may be accessed via an opening formed by a transition section of the at least one integrated feature;
the at least one modification comprises passing the assembly component through a window formed by the at least one integrated feature;
the at least one modification comprises passing a portion of the suture through the window formed by the at least one integrated feature;
passing a portion of the suture or an assembly component into the opening formed by the transition section of the at least one integrated feature of the suture;
the passing includes splicing an end portion of the suture or the assembly component into the central portion of the suture;
the passing extends through the central portion of the suture between two integrated features extending from a first integrated feature at the first predetermined length to a second integrated feature at a second predetermined length;
the at least one modification comprises trimming a portion of the suture at a transition section formed by the at least one integrated feature;
the trimming of the portion of the suture is located at a second predetermined length of a second integrated feature positioned at a predetermined spacing relative to a first integrated feature;
the assembly component comprises at least one of an additional suture, a stuffing or filling material, a rigid or semi-rigid core, an anchor, an implant, a connector, or a pledget;
the at least one integrated feature comprises a plurality of integrated features each positioned at a predetermined length of the suture or a predetermined spacing relative to one or more of the plurality of integrated features;
the plurality of integrated features comprise a first integrated feature positioned at the first predetermined length and a second integrated feature positioned at a second predetermined length;
the at least one modification is completed by modifying the suture at the second predetermined length in response to identifying the second integrated feature thereby forming the surgical construct by interfacing with the at least one integrated feature;
the plurality of integrated features comprise a third integrated feature positioned at a third predetermined length;
the at least one modification is completed by modifying the suture at the third predetermined length in response to identifying the third integrated feature thereby forming the surgical construct by interfacing with the at least one integrated feature; and/or
the predetermined nature of the predetermined length indicates that the corresponding at least one integrated features is positioned at a known position along a length of the suture relative to an overall length of the suture or the position of an additional one of the at least one integrated features.

As used herein, words of approximation such as, without limitation, “approximately,” “substantially,” or “about” refer to a condition that when so modified is understood to not necessarily be absolute or perfect but would be considered close enough to those of ordinary skill in the art to warrant designating the condition as being present. Further, the lack of such modifying terms does not otherwise require strict interpretation of the corresponding value or property. Instead, the extent to which the associated interpretation varies will depend on how great a change can be instituted and still have one of ordinary skill in the art recognize the modified feature as having the required characteristics or capabilities of the unmodified feature. Such determinations may vary considerably depending on the technological field based on the corresponding equivalency associated with the described operation or property. In general, but subject to the preceding discussion, a numerical value herein that is modified by a word of approximation such as “approximately” may vary from the stated value by ±0.5%, ±1%, ±2%, ±3%, ±4%, ±5%, ±10%, ±12%, or ±15%.

Any element in a claim that does not explicitly state “means” for performing a specified function or “step” for performing a specified function, should not be interpreted as a “means” or “step” clause as specified in 35 U.S.C. § 112.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents.

1. A suture formed from a plurality of continuous strands comprising:
a continuous length of a carrier braid extending from a first end portion to a second end portion; and
a plurality of windows comprising a plurality of legs bifurcated from the continuous strands and defining at least one window length.

2. The suture according to claim 1, wherein the windows are separated by a bridge length of a window bridge of the suture.

3. The suture according to claim 2, wherein the bridge length of the window bridge is between 0.5 mm and 128 mm.

4. The suture according to claim 2, wherein the bridge length defines a splicing region of the carrier braid configured to receive and pass a length of suture.

5. The suture according to claim 1, wherein the plurality of windows comprises three or more windows separated by a plurality of window bridges extending along the length.

6. The suture according to claim 1, wherein the windows comprise a first window extending over a first window length and a second window extending over a second window length, wherein the first window length is greater than the second window length.

7. The suture according to claim 1, wherein the at least one window length of the windows is between 0.5 mm to 40 mm.

8. The suture according to claim 1, wherein a strand density of the windows is between 10 and 300 picks per inch (PPI).

9. The suture according to claim 1, wherein the continuous length of the carrier braid comprises a cross-section of a round shape, a rectangular shape, or a ribbon-like shape.

10. The suture according to claim 9, wherein the continuous strands forming the round shape or the rectangular shape form sidewalls that are bifurcated, forming the legs of the windows.

11. The suture according to claim 10, wherein the windows each form an opening to a central portion of the cross-section of the carrier braid.

12. The suture according to claim 11, wherein the openings in the central portion are configured to receive and pass a shuttle suture through a window bridge extending over a bridge length between the plurality of windows.

13. The suture according to claim 1, wherein the legs forming at least one of the windows comprise an equal number of the continuous strands.

14. The suture according to claim 13, wherein a first strand of the continuous strands forming the first leg is a first color, and a second strand of the continuous strands forming the second leg is a second color, different from the first color.

15. The suture according to claim 1, wherein the legs comprise a first leg and a second leg, wherein the first leg comprises more of the continuous strands than the second leg.

16. A method for providing a suture with an automated braiding machine, the method comprising:
braiding a carrier suture from a length of continuous strands extending from a first end portion to a second end portion; and
bifurcating the continuous strands along a pair of legs defining a plurality of windows comprising a plurality of legs from the continuous strands at least one window length.

17. The method according to claim 16, wherein defining the plurality of windows comprises joining the legs between the plurality of windows forming a window bridge over a bridge length.

18. The method according to claim 16, wherein the legs of the windows are formed from sidewalls of the carrier braid forming a round shape or a rectangular shape form.

19. The method according to claim 16, further comprising:
bifurcating the continuous strands of the carrier suture into a split section comprising a first tail and a second tail forming the second end portion.

20. A suture formed from a plurality of continuous strands comprising:
a continuous length of a carrier braid extending from a first end portion to a second end portion; and
a plurality of transition sections of the carrier braid from a round cross-section to a flat or ribbon-like cross-section, wherein the plurality of transition sections comprise opposing, adjacent round to flat transitions that form an opening to a central portion of the cross-section of the carrier braid, wherein the plurality of transition sections along the continuous length define a splicing region over a splice length between the transition sections forming the grouped transition, the splicing region configured to pass a suture or insert between the transition sections.
